(19) United States
(12) Patent Application Publication
(10) Pub. No.: US 2022/0211867 A1
(43) Pub. Date: Jul. 7, 2022

(54) NANOPARTICLE FOR SPECIFICALLY HYDROLYZING TEMPLATE PROTEIN MOLECULE, AND PREPARATION AND APPLICATION THEREOF

(71) Applicant: Guangzhou University of Chinese Medicine (Guangzhou Institute of Traditional Chinese Medicine), Guangzhou (CN)

(72) Inventors: Shixia GUAN, Guangzhou (CN); Liuting ZHONG, Guangzhou (CN)

(21) Appl. No.: 17/703,946

(22) Filed: Mar. 24, 2022

(30) Foreign Application Priority Data
Mar. 30, 2021 (CN) 202110339263.5

(51) Int. Cl. A61K 47/69; A61K 38/48; A61K 47/60; A61K 9/51; A61P 29/00; A61P 37/02

(52) U.S. Cl. CPC A61K 47/6925; A61K 38/482; A61K 47/60; C12Y 304/21106; A61K 9/5192; A61P 29/00; A61P 37/02; A61K 9/5146

(57) ABSTRACT

Disclosed are a nanoparticle for specifically hydrolyzing a template protein molecule, and a preparation and application thereof. The nanoparticle includes a nanozyme as a core and a template protein-imprinted polymer as a shell The nanoparticle can be used in the preparation of drugs for treating cytokine release syndrome.

### CROSS-REFERENCE TO RELATED APPLICATIONS

This application claims the benefit of priority from Chinese Patent Application No. 202110339263.5, filed on Mar. 30, 2021. The content of the aforementioned applications, including any intervening amendments thereto, is incorporated herein by reference in its entirety.

### TECHNICAL FIELD

This application relates to pharmaceutics, and more particularity to a nanoparticle for specifically hydrolyzing a template protein molecule, and a preparation and application thereof.

### BACKGROUND

Cytokine release syndrome (CRS) is a fatal uncontrolled systemic inflammatory response, which is triggered by an acute inflammatory response and characterized by fever, hypotension and respiratory insufficiency associated with elevated serum cytokine. The CRS is commonly developed in patients receiving an immune-related biologic therapy. After triggered, the CRS is generally treated in clinical with a combination of tocilizumab and hormones. Nevertheless, for the severe CRS, the immunotherapy will be abandoned. The triggering mechanism of the CRS still remains unknown, and there is a lack of a specific drug in the clinical practice. Though the plasma exchange is feasible to treat the sever CRS, it is too costly.

### SUMMARY

A first object of this application is to provide a nanoparticle for specifically hydrolyzing a template protein molecule, which is capable of regulating the excessively secreted cytokines or proteins in a variety of diseases.

A second object of this application is to provide a method for preparing the above-mentioned nanoparticle.

A third object of this application is to provide a method for treating cytokine release syndrome (CRS) in a subject in need thereof, comprising:

administering to the subject a therapeutically effective amount of the above-mentioned nanoparticle.

The technical solutions of the present disclosure are described as follows.

In a first aspect, this application provides a nanoparticle for specifically hydrolyzing a template protein, wherein the nanoparticle comprises a nanozyme as a core and a template protein-molecularly imprinted polymer as a shell; and

a particle size of the nanoparticle is 1 nm-50 m, preferably 100 nm-5 μm.

In some embodiments, an enzyme of the nanozyme is serine proteinase;

the serine proteinase is human neutrophil elastase, cathepsin G, protease 3 or a combination thereof;

the nanozyme further comprises a water-insoluble carrier;

the water-insoluble carrier is an inorganic salt crystal;

the inorganic salt crystal is a copper phosphate crystal, a calcium hydrogen phosphate crystal or a combination thereof; and

the nanozyme has an inorganic hybrid nanoflower structure formed by hybridization of the enzyme with the inorganic salt crystal.

In some embodiments, a raw material for preparing the template protein-molecularly imprinted polymer comprises an organic polymer material and the template protein.

In some embodiments, the organic polymer material is a positively-charged amino-rich material.

In some embodiments, the organic polymer material is a water-soluble polysaccharide.

In some embodiments, the template protein is a cytokine, a coagulation factor, an immunoglobulin, a complement or a protein from an extracellular matrix.

In some embodiments, the protein is collage, elastin, fibrin, fibronectin or a combination thereof.

In some embodiments, the cytokine is interleukin, interferon, tumor necrosis factor superfamily, colony-stimulating factor, chemokine, growth factor or a combination thereof.

In some embodiments, the interleukin is interleukin-6 (IL-6), IL-2, IL-8 or a combination thereof.

In some embodiments, the raw material for preparing the template protein-molecularly imprinted polymer further comprises dopamine.

In some embodiments, the shell of the nanoparticle is a polydopamine layer wrapped on a surface of the nanozyme; and there is a cavity between the nanozyme and the polydopamine layer.

In some embodiments, the nanozyme has a nanoflower structure formed by hybridization of the human neutrophil elastase with the copper phosphate crystal.

In some embodiments, a surface of the nanoparticle is provided with a targeted modification material; and the targeted modification material is polyethylene glycol.

In a second aspect, this application provides a method for preparing the above-mentioned nanoparticle, comprising:

wrapping a template protein-molecularly imprinted polymer on a surface of a nanozyme to form the nanoparticle with a core-shell structure.

In some embodiments, the method comprises:

coating an organic polymer material layer on the surface of the nanozyme; allowing a template protein to be adsorbed to a surface of the organic polymer material layer; and preparing a polydopamine layer on the organic polymer material layer by polymerization.

In an embodiment, the method further comprises:

after the polydopamine layer is prepared, removing the template protein and the organic polymer material layer to form a cavity between the nanozyme and the polydopamine layer.

In an embodiment, a preparation of the nanozyme comprises:

subjecting a human neutrophil elastase and a copper phosphate crystal to hybridization to form the nanozyme with an inorganic hybrid nanoflower structure.

In an embodiment, the preparation of the nanozyme comprises:

reacting the human neutrophil elastase with an aqueous copper sulphate solution in a phosphate buffered saline (PBS) containing bovine serum albumin (BSA) followed by solid-liquid separation to collect the nanozyme.

In some embodiments, the method further comprises:

dispersing the nanozyme in a chitosan solution to obtain a chitosan-coated nanozyme;

dispersing the chitosan-coated nanozyme in a Tris buffer followed by mixing with IL-6 to allow the IL-6 to be adsorbed on a surface of the chitosan-coated nanozyme; and

mixing the chitosan-coated nanozyme adsorbed with the IL-6 with a dopamine solution followed by polymerization and solid-liquid separation to collect a solid.

In an embodiment, the method further comprises:

subjecting the solid to elution and dialysis to remove chitosan and the IL-6.

In an embodiment, the method further comprises:

subjecting a surface of the nanoparticle to targeted modification.

In a third aspect, this application also provides a method for treating cytokine release syndrome in a subject in need thereof, comprising:

administering to the subject a therapeutically effective amount of the above-mentioned nanoparticle.

This application has the following beneficial effects.

The nanoparticle provided herein includes a nanozyme as a core and a template protein-molecularly imprinted polymer as a shell. The nanoparticle has a strong applicability, and can regulate the excessively secreted cytokines or proteins in a variety of diseases to enable a better therapeutic effect and relieve the adverse effect of drugs. The preparation method of the nanoparticle has simple operation, and the nanoparticle can specifically identify, bind and hydrolyze cytokine. The nanoparticle provided herein can be applied to the preparation of drugs for the treatment of CRS, and has broad application prospects.

### BRIEF DESCRIPTION OF THE DRAWINGS

The accompany drawings needed to be used in the embodiments will be briefly introduced below to more clearly explain the technical solutions of the present disclosure and the prior art. Obviously, illustrated in the accompany drawings are merely embodiments of the present disclosure, which are not intended to limit the present disclosure. Other drawings can be obtained by those skilled in the art based on the drawings provided herein without sparing creative effort.

FIG. 1 shows zeta potentials of nanoparticles prepared in Example 2;

FIG. 2 shows Fourier transform infrared (FT-IR) spectra of the nanoparticles prepared in Example 2;

FIGS. 3A-3B show scanning electron microscopy (SEM) images of the nanoparticles prepared in Example 2;

FIG. 4 shows X-ray photoelectron spectroscopy (XPS) analysis results of the nanoparticles prepared in Example 2;

FIGS. 5A-5D show XPS peak-differentiation-imitating analysis results of C1s of the nanoparticles prepared in Example 2;

FIG. 6 shows hydrolysis results of IL-6 respectively under the catalysis of free enzymes, BSA-Cu₃(PO₄)₂.3H₂O hybrid nanozymes (NCs) and chitosan-coated BSA-Cu₃(PO₄)₂.3H₂O hybrid NCs (CS-NCs) in Example 3;

FIG. 7 shows catalyzed hydrolysis results of IL-6 under the catalysis of a molecularly imprinted polymer (MIP) in Example 3;

FIG. 8 shows electrophoretograms of hydrolysis products of IL-6 under the catalysis of MIP after 3 cycles in Example 3;

FIG. 9 shows a relationship between the MIP-catalyzed hydrolysis effect of IL-6 and a template protein level during the imprinting process in Example 4;

FIGS. 10A-10B show electrophoretograms and gray values of hydrolysis products of IL-6 under the catalysis of different nanoparticles in Example 5, where 10A: hydrolyzed for 24 h; and 10B: hydrolyzed for 48 h;

FIG. 11 illustrates cytotoxicity assay results of MIP and non-imprinted polymer (NIP) in Example 6;

FIG. 12A shows a change of NO level with the lipopolysaccharide (LPS) concentration;

FIG. 12B shows a change of IL-6 level with the LPS concentration;

FIGS. 13A-13D show an effect of the co-culture of cells and MIP on the hydrolysis of IL-6 in Embodiment 8, where 13A: 0.01 mg/mL MIP; 13B: 0.025 mg/mL MIP; 13C: 0.05 mg/mL; and 13D: 0.1 mg/mL MIP;

FIGS. 14A-14H show hematologic parameter analysis results of MIP-treated mice in Example 9, where 14A: white blood cell count (WBC); 14B: lymph %; 14C: the percentage of monocytes (Mon %); 14D: the percentage of neutrophil granulocytes (Gran %); 14E: red blood cell count (RBC); 14F: hemoglobin (HGB): 14G: platelet count (PLT); and 14H: mean platelet volume (MPV);

FIGS. 15A-15E show liver function parameter and renal function parameter test results of the MIP-treated mice in Example 9, where 15A: alanine transaminase (ALT); 15B: aspartate aminotransferase (AST); 15C: blood urea nitrogen (BUN); 15D: creatinine (CREA); and 15E: uric acid (UA);

FIG. 16 shows the inflammatory cytokine level of the MIP-treated mice in Example 9;

FIGS. 17A-17H show hematologic parameter analysis results of mice from different groups in Example 9, where 17A: WBC; 17B: Lymph %; 17C: Mon %; 17D: Gran %; 17E: RBC; 17F: HGB; 17G: PLT; and 17H: MPV;

FIGS. 18A-18E show liver function parameter and renal function parameter test results of the mice from different groups in Example 9, where 18A: ALT; 18B: AST; 18C: BUN; 18D: CREA; and 18E: UA;

FIGS. 19A-19C show the level of three kinds of inflammatory cytokines in the mice from different groups in Example 9, where 19A: IL-6; 19B: IL-8; and 19C: TNF-α;

FIGS. 20A-20B show the distribution of 1,1-dioctadecyl-3,3,3,3-tetramethylindotricarbocyanine iodide (DIR) in nude mice, where 20A: back; and 20B: abdomen; and

FIGS. 20C-20D show the level of DIR in different organs of the nude mice.

### DETAILED DESCRIPTION OF EMBODIMENTS

In order to make the objects, advantages and technical solutions of the present disclosure clearer, the present disclosure will be clearly and completely described below with reference to the embodiments and accompanying drawings. Unless otherwise specified, the Examples are carried out in conventional conditions or as recommended by the manufacturer, and the materials, reagents and instruments adopted in the following embodiments are commercially available.

The nanoparticle provided herein for specifically hydrolyzing a template protein molecule, and a preparation and application thereof will be specifically described below.

The disclosure provides a nanoparticle for specifically hydrolyzing a template protein molecule, which includes a nanozyme as a core and a template protein-molecularly imprinted polymer as a shell.

A particle size of the nanoparticle is 1 nm-50 m, preferably 100 nm-5 μm.

The nanozyme includes an enzyme. In an embodiment, the enzyme is serine proteinase.

The serine proteinase is human neutrophil elastase (HNE), cathepsin G (CG), protease 3 (PR3) or a combination thereof.

The nanozyme further includes a water-insoluble carrier, such as an inorganic salt crystal. In an embodiment, the inorganic salt crystal is a copper phosphate crystal, a calcium hydrogen phosphate crystal or a combination thereof.

In an embodiment, the nanozyme provided herein is a nano-sized enzyme with an inorganic hybrid nanoflower structure formed by hybridization of the enzyme with the inorganic salt crystal, and has a high enzymatic activity and stability.

A raw material for preparing the template protein-molecularly imprinted polymer includes an organic polymer material and a template protein.

The organic polymer material can form an inert gel which can be easily activated to reversibly and irreversibly bind to proteins and enzymes. In an embodiment, the organic polymer material is an amino-rich positively-charged material. In an embodiment, the organic polymer material is a water-soluble polysaccharide, such as carrageenan, chitosan, sodium alginate, cellulose, agarose and starch.

The template protein is cytokine (CK), coagulation factor, immunoglobulin, complement or a protein from an extracellular matrix, such as collage, elastin, fibrin, fibronectin and a combination thereof.

The CK is a low-molecular weight soluble protein produced by a variety of cells under the induction of immunogens, mitogens or other stimulants, and plays an important role in the regulation of innate and adaptive immunity, hematopoiesis, cell growth, the differentiation of APSC pluripotent cells and repair of damaged tissues.

The cytokine is interleukin (IL), interferon, tumor necrosis factor superfamily, colony-stimulating factor, chemokine, growth factor or a combination thereof.

The CK is selected from the group consisting of IL-1, IL-2, IL-6, IL-7, IL-8, IL-10, IL-12, tumor necrosis factor-α (TNF-α), interferon-α (IFN-α), IFN-β, IFN-γ, macrophage chemoattractant protein-1 (MCP-1) and C-X-C motif chemokine 10 (CXCL10).

The raw material for preparing the template protein-molecularly imprinted polymer further includes dopamine. The dopamine can form polydopamine under alkaline conditions through oxidative polymerization. A surface of the polydopamine is rich in free amino and carboxyl groups, and is negatively-charged.

Since a surface of the nanozyme is negatively-charged and a surface of the organic polymer material is positively-charged, by means of the electrostatic attraction of positive and negative charges, the organic polymer material can be coated on the surface of the nanozyme to obtain an organic polymer-coated nanozyme with the surface positively-charged. Consequently, the negatively-charged polydopamine can be bound to the organic polymer-coated nanozyme.

It should be noted that the nanoparticle provided here has two structural forms, where the first structure contains the nanozyme, organic polymer material, polydopamine, and template protein; and the second structure is merely composed of the nanozyme and polydopamine.

It can be understood as that the first structure includes a core (i.e., the nanozyme) and a shell, where the shell includes an organic polymer material layer coated on the nanozyme and a polydopamine layer polymerized on a surface of the organic polymer layer, and the template protein molecule (such as CK) is adsorbed on the surface of the organic polymer layer; the second structure includes a core (i.e., the nanozyme) and a shell, where the shell is a polydopamine layer coated on the surface of the nanozyme, and there is a cavity between the nanozyme and the polydopamine layer.

In an embodiment, with respect to the first structure, the core is the nanozyme with the hybrid nanoflower structure formed by hybridization of the human neutrophil elastase with the copper phosphate crystal. The shell includes a chitosan layer coated on the nanozyme and a polydopamine layer formed on a surface of the chitosan layer. The IL-6 is adsorbed on the surface of the chitosan layer. With respect to the second structure, the core is the nanozyme with the hybrid nanoflower structure formed by hybridization of the human neutrophil elastase with the copper phosphate crystal. The shell in the second structure is a polydopamine layer. There is a cavity between the nanozyme and the polydopamine layer.

In an embodiment, a surface of the nanoparticle further includes a targeted modification material, such as polyethylene glycol (PEG) or other common targeted modification materials. The nanoparticle can target different organs, and by means of the modification with a specific ligand, especially PEG, the long circulation can be achieved.

This application provides a method for preparing the above-mentioned nanoparticle, including:

coating the template protein-molecularly imprinted polymer on the surface of the nanozyme to form the nanoparticle with the core-shell structure.

With respect to the first structure, the organic polymer material layer is coated on the surface of the nanozyme. The template protein molecule is adsorbed to the surface of the organic polymer material layer. The polydopamine layer is formed on the organic polymer material layer by polymerization.

With respect to the second structure, after the polydopamine layer is formed, the template protein molecule and the organic polymer material layer are removed to form the cavity between the nanozyme and the polydopamine layer.

In an embodiment, a hybridization of the human neutrophil elastase with the copper phosphate crystal is performed to form the nanozyme with the inorganic hybrid nanoflower structure. Optionally, the HNE and an aqueous copper sulphate (CuSO₄) solution are mixed in a BSA-containing PBS and reacted, and the reaction product is subjected to solid-liquid separation to collect the nanozyme.

In the above-mentioned process, the BSA, HNE and copper ion (Cu²⁺) form a complex, which becomes nucleation sites for the primary copper phosphate crystal. The interaction between BSA, HNE and Cu²⁺ contributes to the growth of micron-sized particles with a nanoflower structure. Compared to free enzymes, the proteases exhibit a higher enzymatic activity and stability.

In an embodiment, in the preparation method of the nanoparticle, the nanozyme is dispersed in a chitosan solution to obtain a chitosan-coated nanozyme, which is dispersed in a Tris buffer and mixed with IL-6 to allow the IL-6 to be adsorbed to a surface of the chitosan-coated nanozyme. Then the chitosan-coated nanozyme with the IL-6 adsorbed thereto is mixed with a dopamine solution and subjected to polymerization and solid-liquid separation to collect a solid. In an embodiment, the solid is subjected to elution and dialysis to remove the chitosan layer and the IL-6.

The coating with the chitosan layer has a certain masking effect on the nanozyme. The IL-6 can be imprinted on the chitosan-nanozyme material by molecular imprinting, and then removed by elution. According to the electrostatic adsorption, most of the chitosan coated on the surface of the nanozyme is removed by dialysis to generate the small cavity between the nanozyme and the polydopamine layer. By means of the inherent selectivity of the molecular imprinting, the IL-6 can enter into an imprinted site and further diffuse into the small cavity to be fully exposed to the nanozyme. The IL-6 is hydrolyzed into multiple fragments under the catalysis of the nanozyme, which diffuse out of the molecularly imprinted polymer through the imprinted site. Therefore, the remaining IL-6 molecules can enter the nanoparticle to allow the continuous hydrolysis under the catalysis of the nanozyme, namely, the nanoparticle can be reused. After repeated utilization, the nanoparticle still maintains a relatively high enzymatic activity.

In an embodiment, the surface of the nanoparticle is subjected to targeted modification to allow the organ targeting and long circulation.

The specific preparation of the nanoparticle is described as follows.

(S1) 1 mL of PBS containing 0.5 mg/mL of BSA was mixed evenly with 20 μL of 0.5 mg/mL HNE and an aqueous CuSO₄ solution (final concentration: 1.6 mM), and reacted at 37° C. under shaking for 2 h. The reaction mixture was centrifuged at 10,000 rpm for 5 min to obtain the precipitate, which was subjected to centrifugal washing 3 times with deionized water. A supernatant was discarded to obtain BSA-Cu₃(PO₄)₂.3H₂O hybrid nanozymes (NCs). The NCs were dispersed in 1 mL of 0.1 M Tris-HCl, 0.5 M NaCl (pH 7.5) buffer.

(S2) The NCs obtained in step (S1) were dispersed in 1 mL of 1.15 mg/mL chitosan (CS) solution (pH 6.2), stirred for 30 min and subjected to centrifugal washing (10000 rpm, 5 min) once with deionized water. A supernatant was discarded to obtain chitosan-coated BSA-Cu₃(PO₄)₂.3H₂O hybrid NCs (CS-NCs). The CS-NCs were dispersed in 1 mL of 0.1 M Tris-HCl, 0.5 M NaCl (pH 7.5) buffer.

(S3) The CS-NCs obtained in step (S2) were dispersed in 20 mM Tris buffer (pH 8.0) to obtain a CS-NC solution. 1 mL of the CS-NC solution was evenly mixed with L of 0.1 mg/mL IL-6 solution and incubated at 37° C. for 1 h. The reaction mixture was added with 0.1 mg/mL dopamine (DA) solution, reacted at 37° C. under shaking for 2.5 h and centrifuged at 10,000 rpm for 10 min. The supernatant was discarded, and the precipitate was subjected to elution with 1 mL of 0.1 M Tris, 1 M NaCl (pH 8.0) buffer and ultrasonic dispersion in ice water bath for 30 min, and this process was repeated 3 times. Then the reaction mixture was centrifuged at 10,000 rpm for 10 min to obtain a polydopamine-CS-NCs conjugate (PDA@CS-NCs).

(S4) The PDA@CS-NCs obtained in step (S3) was loaded into a dialysis bag (300000, Spectrum Inc., US) dialyzed in 500 mL of 0.1 M Tris, 1 M NaCl (pH 5.5) buffer overnight. The dialysis solution was replaced for another 6 h dialysis. Then the sample was removed and centrifuged at 10,000 rpm for 5 min. The supernatant was discarded, and the precipitate was a molecularly imprinted polymer (MIP). The MIP was dispersed in 1 mL of 0.1 M Tris, 0.5 M NaCl (pH 7.5) buffer.

The PDA@CS-NCs and the MIP respectively obtained in steps (S3) and (S4) both fall within the scope of the present disclosure defined by the appended claims.

In an embodiment, a concentration of the aqueous CuSO₄ solution in step (S1) is 0.8 mM-1.6 mM, such as 0.8 mM, 1 mM and 1.5 mM, and the pH can be adjusted to 7.5-8.

It should be noted that nanoparticles prepared from other enzymes, organic polymer materials, template protein molecules and polydopamine also fall within the scope of the disclosure, and the preparation process can be adjusted according to the actual situation and is not specifically limited here.

In addition, this application provides an application of the nanoparticle in the preparation of a drug for the treatment of cytokine release syndrome (CRS).

Among the cytokines, the IL-6 is a desirable indicator to evaluate the severity and prognosis of most diseases manifested by cytokine storm, and is a suitable target molecule for the cytokine storm. With respect to the nanoparticle prepared with IL-6 as the cytokine molecule, it can specifically catalyze the hydrolysis of IL-6 to reduce the IL-6 level to prevent the occurrence of CRS or attenuate the inflammatory response when the CRS is triggered. It is worth noting that in the prior art, the tocilizumab is used to block the IL-6-induced signal transduction pathway by competitively inhibiting the binding of IL-6 binding to the receptor, so as to alleviate the CRS. Differently, the nanoparticle provided herein aims to catalyze the degradation of excessively secreted IL-6. By means of the catalytic activity of enzymes and the molecular imprinting technique, the nanoparticle can specifically recognize the IL-6 and catalyze the hydrolysis of the IL-6 to timely remove the excessively secreted IL-6 to prevent the CRS from being triggered.

The features and performances of the disclosure will be further described below with reference to embodiments.

### Example 1

Provided herein was a nanoparticle, which was prepared through the following steps.

(S1) Preparation of NCs

1 mL of PBS containing 0.5 mg/mL BSA was mixed evenly with 20 μL of 0.5 mg/mL HNE and an aqueous CuSO₄ solution (final concentration: 1.6 mM) and reacted at 37° C. under shaking for 2 h. The reaction mixture was centrifuged at 10,000 rpm for 5 min to obtain the precipitate, which was subjected to centrifugal washing 3 times with deionized water. The precipitate was collected as the NCs. The NCs were dispersed in 1 mL of 0.1 M Tris-HCl, 0.5 M NaCl (pH 7.5) buffer.

(S2) The NCs obtained in step (S1) were dispersed in 1 mL of 1.15 mg/mL CS solution (pH 6.2), stirred for 30 min and subjected to centrifugal washing (10000 rpm, 5 min) once with deionized water. A supernatant was discarded to obtain the CS-NCs. The CS-NCs were dispersed in 1 mL of 0.1 M Tris-HCl, 0.5 M NaCl (pH 7.5) Buffer. The pKₐ of the chitosan molecule is 6.3.

(S3) The CS-NCs obtained in step (S2) were dispersed in 20 mM Tris buffer (pH 8.0) to obtain a CS-NCs solution. 1 mL of the CS-NCs solution was evenly mixed with 10 μL of 0.1 mg/mL IL-6 and incubated at 37° C. for 1 h. The reaction mixture was added with 0.1 mg/mL DA solution, reacted at 37° C. under shaking for 2.5 h and centrifuged at 10,000 rpm for 10 min. The supernatant was discarded, and the precipitate was subjected to elution with 1 mL of 0.1 M Tris, 1 M NaCl (pH 8.0) buffer and ultrasonic dispersion in ice water bath for 30 min, and this process was repeated 3 times. Then the reaction mixture was centrifuged at 10,000 rpm for 10 min to obtain the PDA@CS-NCs.

(S4) The PDA@CS-NCs obtained in step (S3) was loaded into a dialysis bag (300000, Spectrum Inc., US) dialyzed in 500 mL of 0.1 M Tris, 1 M NaCl (pH 5.5) buffer overnight. The dialysis solution was replaced for another 6 h dialysis. Then the sample was removed and centrifuged at 10,000 rpm for 5 min. The supernatant was discarded, and the precipitate was the MIP The MIP was dispersed in 1 mL of 0.1 M Tris, 0.5 M NaCl (pH 7.5) buffer.

### Example 2

### 1. Zeta Potential

The nanoparticle (MIP) obtained in the Example 1 was ultrasonically dispersed in PBS for 5 min to obtain a dispersion with a concentration of 0.05 mg/mL, which was then measured using a Malvern particle size analyzer for the Zeta potential. Meanwhile, Zeta potentials of the NCs, CS-NCs, PDA@CS-NCs and a non-imprinted polymer (NIP) were analyzed.

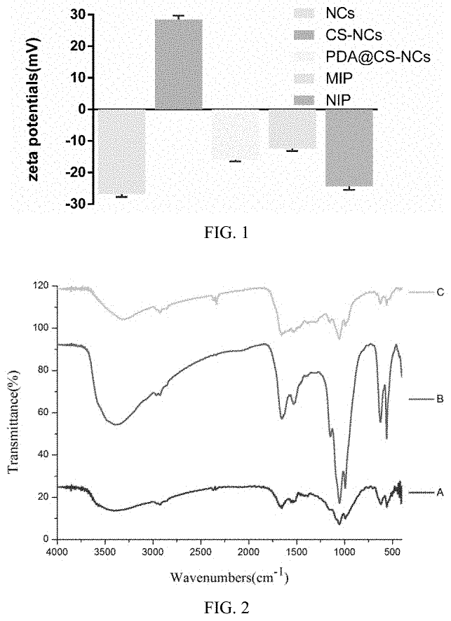

The measurement results were shown in FIG. 1. It can be observed that the NCs had a negative-charged surface, and after coated with the chitosan layer, the surface of the NCs was reversed to be positively-charged. The dopamine underwent oxidative polymerization under alkaline conditions to form the negatively-charged polydopamine. The CS-NCs was sequentially added with the template IL-6 and the dopamine for oxidative polymerization, and subjected to elution to remove the template IL-6, so as to obtain the PDA@CS-NCs. The PDA@CS-NCs was subjected to dialysis to obtain the MIP The molecularly imprinted polymer prepared in the absence of the template IL-6 was named as non-imprinted polymer (NIP). The NIP was obtained by polymerizing a polydopamine film on a surface of the CS-NCs, and thus its surface carried a large number of negative charges with a potential of about −25 mV After the template removal, the imprinted site was left in the PDA@CS-NCs, such that the chitosan might be partially exposed to neutralize some negative charges, leading to an increase in the potential (about −16 mV). The chitosan was mostly removed by dialysis, and the obtained MIP had a potential of around −12.5 mV.

### 2. Fourier Transform Infrared Spectrometry (FT-IR)

The sample was treated by potassium bromide pellet technique and analyzed using a Fourier transform infrared spectrometer. Specifically, 1-2 mg of a vacuum-dried sample was mixed with 100 mg of powdered KBr in an agate mortar. The mixture was ground in the same direction and pressed into a tablet. The tablet was transferred to the Fourier transform infrared spectrometer for analysis, where the wavenumber range was 4000-400 cm⁻¹ and the resolution was 0.5 cm⁻¹.

The FT-IR results were shown in FIG. 2, where (A) CS-NCs; (B) polydopamine coated NCs (PDA@NCs); and (C) PDA@CS-NCs. The strong absorption at 1100-1050 cm⁻¹ and 1000-970 cm⁻¹ indicated the asymmetric and symmetric stretching vibration of PO₄³⁻; the weak absorption at 650-610 cm⁻¹ and 580-540 cm⁻¹ indicated the asymmetric and symmetric stretching vibration of PO₄³⁻, which demonstrated the presence of phosphate. Since the chitosan molecule and the polydopamine both contained hydroxyl and amino groups, functional groups of the CS-NCs (A) were identical to those of PDA@NCs (B), and there was no obvious chemical shift therebetween. The absorption at 3500-3300 cm⁻¹ indicated the stretching vibration of —OH; the absorption at 3000-2850 cm⁻¹ indicated the stretching vibration of —CH₂ and —CH₃; the absorption band of the amide I existed at 1680-1655 cm⁻¹; the absorption band of the amide II existed at 1550-1535 cm⁻¹; the absorption band of amide III located at 1300-1260 cm⁻¹; and the adsorption at 1350-1250 cm⁻¹ indicated a stretching vibration of —CN. Additionally, no new absorption peaks or large chemical shifts were observed for the PDA@CS-NCs obtained sequentially by chitosan coating and dopamine polymerization, suggesting that structures of the chitosan, polydopamine and NCs essentially remained unchanged.

### 3. Scanning Electron Microscopy (SEM)

A small amount of vacuum-dried sample powder was spread on a conductive adhesive of a sample plate, subjected to vacuum gold spraying and observed under a scanning electron microscope for the surface and morphology.

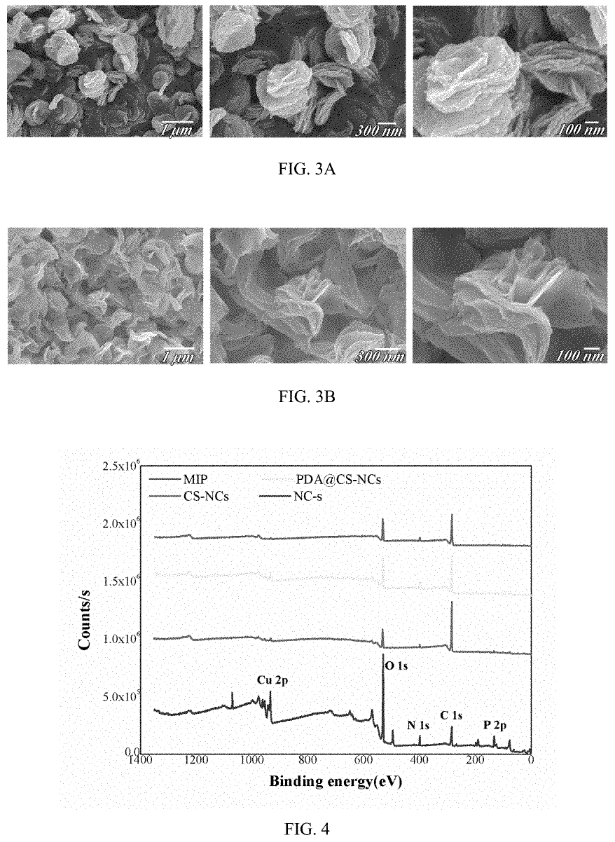

The SEM images of the CS-NCs were shown in FIG. 3A, and the SEM images of the PDA@CS-NCs were shown in FIG. 3B. Compared to the NCs, the CS-NCs had an increased surface roughness, which might be caused by the adsorption of the chitosan on the surface of the NCs. Compared to the CS-NCs, the PDA@CS-NCs had a slighter roughness, which might be caused by the formation of the polydopamine film on the surface.

### 4. X-Ray Photoelectron Spectroscopy (XPS)

The sample was analyzed under vacuum at a pass energy of 150 eV (measurement scan) or 25 eV (high-resolution scan) by means of a Thermo Scientific™ K-Alpha™ spectrometer equipped with a monochromatic Al KαX-ray source (1486.6 eV). With respect to adventitious carbons, all peaks were calibrated with a C1s peak binding energy of 284.8 eV All peaks were fitted by Avantage.

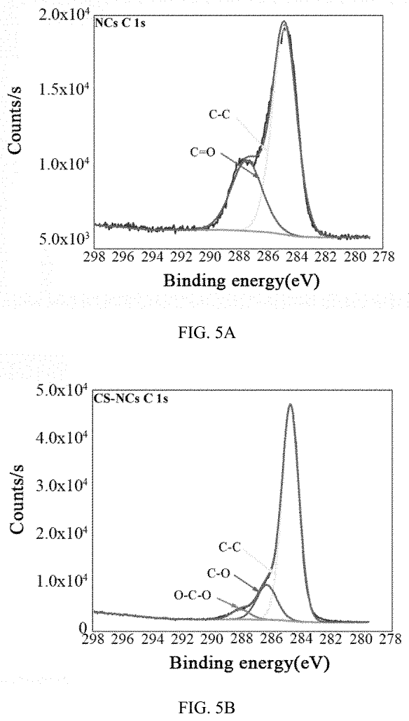

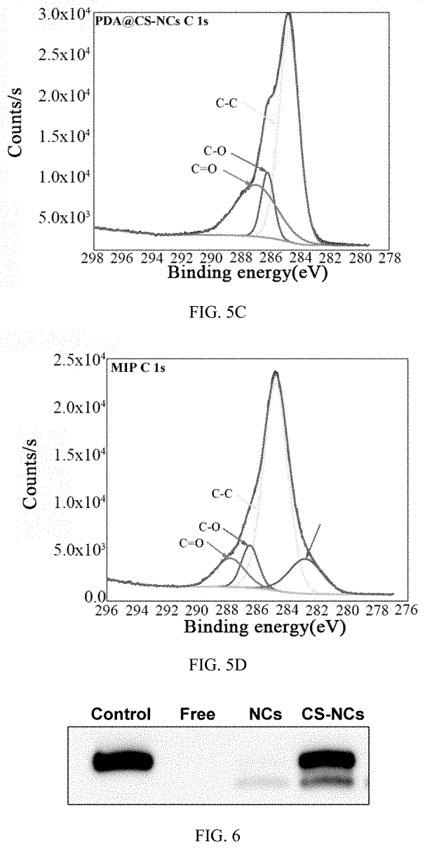

FIG. 4 displayed the XPS characterization results of the NCs, CS-NCs, PDA@CS-NCs and MIP FIGS. 5A-5D respectively illustrated peak-differentiation-imitating analysis results of the C1s of the NCs, CS-NCs, PDA@CS-NCs and MIP. The element compositions (C, N, O, P, and Cu) of surfaces of the four samples were presented in Tables. 1-4, respectively.

As shown in FIG. 4, the NCs exhibited obvious Cu 2p, P 2p and O 1s peaks, which were consistent with peaks of Cu₃(PO₄)₂.3H₂O. Besides, the NCs also showed obvious C 1s and N 1s peaks, which were derived from the doped BSA/HNE protein. The above five peaks constituted the main characteristic elemental peaks of the nanoflower.

The peak separation of the C 1s peak of the NCs was shown in FIG. 5A. Since the BSA/HNE was immobilized with the copper phosphate by self-assembly rather than covalent bonding, the NCs mainly contained amido bond and C—C from the protein. The C 1s peak of the NCs was fitted by Avantage, and differentiated into two peaks, where the peak at 284.82 eV indicated the C—C, and the peak at 287.42 eV was the C═O in the amido bond.

The CS-NCs, PDA@CS-NCs and MIP were mainly dominated by C, N, O, P and Cu. Since the XPS was merely a surface element analysis tool with a penetration depth of 3-10 nm, and the chitosan merely contained C, N and O, a surface of the CS-NCs mainly contained C, N and O and the proportion of P and Cu was less than 5%. Similarly, surfaces of the PDA@CS-NCs and the MIP were also mainly composed of C, N and O, and the difference mainly lay in the proportions of C, N, and O and the chemical bonding type.

The peak separation of the C 1s of the CS-NCs was illustrated in FIG. 5B. The chitosan was coated around the NCs by means of electrostatic adsorption. The C 1s peak of the CN—NCs was fitted by Avantage, and differentiated into three peaks, where the peak at 284.79 eV was assigned to C—C; the peak at 286.38 eV was assigned to C—O and the peak at 288.13 eV was assigned to O—C—O of the chitosan.

The peak separation of the C 1s of the PDA@CS-NCs was depicted in FIG. 5C. The C 1s peak of the PDA@CS-NCs was fitted by Avantage, and differentiated into three peaks, where the peak at 284.8 eV was assigned to C—C; the peak at 286.23 eV indicated C—O and the peak at 287.02 eV was C═O, which were consistent with the C1s peak of polydopamine reported previously, suggesting that the polydopamine had been successfully coated on the surface of the CS-NCs.

The peak separation of the C 1s of the MIP was displayed in FIG. 5D. The C 1s peak of the MIP was fitted by Avantage, and differentiated into four peaks, where the peak at 284.79 eV was assigned to C—C; the peak at 286.5 eV was C—O and the peak at 287.81 eV was C═O, which were consistent with the above-mentioned peaks of polydopamine. The peak at 282.81 eV cannot be accurately determined, which was created by the cavity between the core and the shell after the elution and dialysis.

TABLE 1
XPS data of NCs

| | Peak | Height | FWHM | | Atomic |
|---|---|---|---|---|---|
| Name | BE | CPS | eV | Area | % |
| P 2p | 133.18 | 8596.21 | 1.91 | 19629.46 | 9.38 |
| C 1s | 284.82 | 13839.91 | 1.97 | 29611.24 | 20.98 |
| C1s Scan A | 287.42 | 4690.35 | 2.53 | 12885.25 | 9.14 |
| N 1s | 399.64 | 7229.95 | 1.77 | 15265.98 | 6.97 |
| O 1s | 530.87 | 68428.01 | 1.74 | 145181.48 | 42.51 |
| Cu 2p | 934.16 | 23028.7 | 4.25 | 231636.07 | 11.02 |

TABLE 2
XPS data of CS-NCs

| | Peak | Height | FWHM | | Atomic |
|---|---|---|---|---|---|
| Name | BE | CPS | eV | Area | % |
| P 2p | 133.54 | 1095.83 | 1.71 | 2619.73 | 1.77 |
| C 1s | 284.79 | 44364.64 | 1.33 | 64156.92 | 64.39 |
| C1s Scan A | 286.38 | 7208.66 | 1.58 | 12318.58 | 12.38 |
| C1s Scan B | 288.13 | 2074.48 | 1.48 | 3328.83 | 3.35 |
| N 1s | 400 | 2042.46 | 1.7 | 5099.09 | 3.3 |
| O 1s | 532.38 | 11605.42 | 2.68 | 33286.67 | 13.83 |
| Cu 2p | 932.96 | 2582.18 | 2.12 | 14690.67 | 0.99 |

TABLE 3
XPS data of PDA@CS-NCs

| | Peak | Height | FWHM | | Atomic |
|---|---|---|---|---|---|
| Name | BE | CPS | eV | Area | % |
| P 2p | 133.51 | 472.74 | 1.94 | 1509.01 | 0.88 |
| C 1s | 284.8 | 25709.57 | 1.59 | 44286.61 | 38.26 |
| C1s Scan A | 286.23 | 7975.29 | 1.02 | 8832.94 | 7.64 |
| C1s Scan B | 287.02 | 6290.47 | 3.36 | 22941.03 | 19.85 |
| N 1s | 399.71 | 5451.8 | 1.93 | 13341.21 | 7.43 |
| O 1s | 532.16 | 21339.77 | 2.91 | 65661.86 | 23.47 |
| Cu 2p | 932.75 | 6706.34 | 2.2 | 42877.27 | 2.48 |

TABLE 4
XPS data of MIP

| | Peak | Height | FWHM | | Atomic |
|---|---|---|---|---|---|
| Name | BE | CPS | eV | Area | % |
| P2p | 140.09 | 99.16 | 0.06 | 591.1 | 0.43 |
| C1s | 284.79 | 21408.24 | 1.93 | 44781.29 | 47.64 |
| C1s Scan A | 286.5 | 4372.97 | 1.33 | 6305.79 | 6.72 |
| C1s Scan B | 282.81 | 3589.69 | 2.6 | 10119.33 | 10.75 |
| C1s Scan C | 287.81 | 2979 | 2.31 | 7467.14 | 7.96 |
| N1s | 399.61 | 2640.95 | 2.3 | 8078.74 | 5.54 |
| O1s | 532.18 | 14986.11 | 2.73 | 46732.78 | 20.57 |
| Cu2p | 932.86 | 948.62 | 2.32 | 5563.88 | 0.4 |

### Example 3

### 1. Catalytic Activity of NCs and CS-NCs for Hydrolysis of IL-6

To verify a masking effect of the chitosan on the NCs to prevent the NCs from catalyzing the hydrolysis of a substrate, the catalytic activities of free enzymes, NCs and CS-NCs on the hydrolysis of IL-6 were compared, and the results were shown in FIG. 6. It can be concluded that after exposed to the free enzyme for 24 h, the IL-6 was hydrolyzed completely; after exposed to the NCs for 24 h, the IL-6 was mostly hydrolyzed, and a few electrophoretic bands with a molecular weight below 21 kDa were observed, which were polypeptide fragments generated from the hydrolysis. The NCs in the CS-NCs were wrapped and cannot be directly contacted with the IL-6, and thus most of the IL-6 failed to be hydrolyzed and merely a small amount was hydrolyzed into small fragments.

### 2. MIP-Catalyzed Hydrolysis of IL-6

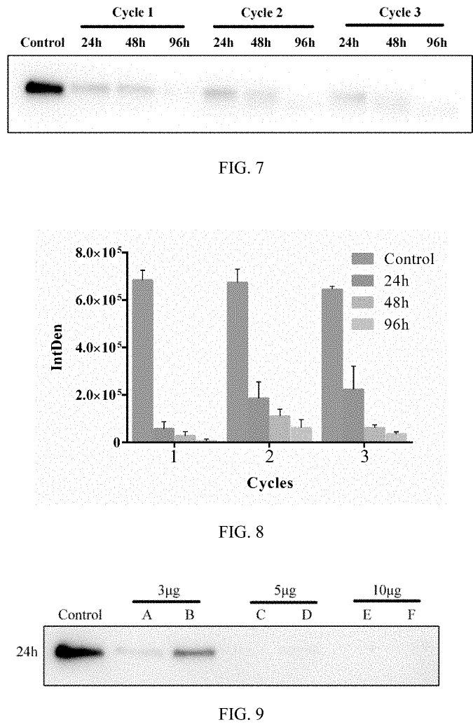

To verify that the MIP was capable of catalyzing the hydrolysis of the IL-6 and the MIP was reusable, the MIP was allowed to be fully reacted with the IL-6, and the results were shown in FIG. 7. Regarding the CS-NCs, the chitosan layer exhibited a masking effect on the NCs. The IL-6 was imprinted on the material by the molecular imprinting. Then the IL-6 was removed by elution, and most of the chitosan wrapped around the surface of the nanozyme by electrostatic adsorption was removed by dialysis. Consequently, there was a cavity formed between the NCs and the polydopamine layer. When the IL-6 bound to the MIP again, by means of the inherent selectivity of the molecular imprinting, the IL-6 entered into the imprinted site and further diffused into the small cavity to be fully exposed to the NCs. The IL-6 was catalytically hydrolyzed into multiple fragments, which diffused out of the MIP through the imprinted site. Therefore, other IL-6 molecules can enter the MIP to allow the NCs to continuously catalyze the hydrolysis of IL-6, indicating that the MIP can be reused.

1 μg of IL-6 was reacted with MIP in a buffer solution for 24 h, 48 h and 96 h, respectively. A supernatant sample after hydrolysis was subjected to electrophoresis. A 96-h reaction was taken as a cycle. The MIP was washed and then reacted with another 1 μg of IL-6 with the same operation to perform a next cycle. A total of 3 cycles were performed. As shown in FIG. 7, the MIP had a role of catalyzing the hydrolysis of IL6. After 3 cycles, the MIP still was capable of catalyzing the IL-6 hydrolysis, suggesting that the MIP was reusable and maintained a high enzymatic activity. Electrophoretic bands of the IL6 hydrolysis products of the 3 cycles were expressed by gray scale, and the results were shown in FIG. 8, which were consistent with the previous results.

### Example 4

The amount of the IL-6 was varied to investigate whether there was a relationship between the catalytic effect of MIP and the amount of template protein added during the imprinting. The results were shown in FIG. 9. The amount of the IL-6 was increased to 3 μg, 5 μg and 10 μg. After 24 h of catalyzed hydrolysis, the efficiency of the catalyzed hydrolysis of 3 μg of IL-6 was similar to that of 1 μg of IL-6. However, when the amount of IL-6 was increased to 5 μg and 10 μg, the hydrolysis efficiency was improved obviously, suggesting that the efficiency of the MIP-catalyzed hydrolysis of IL-6 was related to the amount of template protein added during imprinting.

### Example 5 Selectivity of Molecularly Imprinted Polymer

The preparations of the NCs, CS-NCs, PDA@CS-NCs and MIP were the same with those in Example 1. The preparation of NIP was identical to that in Example 2. A Control group merely contained IL-6.

The NCs, CS-NCs, PDA@CS-NCs, MIP and NIP were respectively dispersed in 1 mL of 0.1MTris, 0.5 M NaCl, pH7.5 buffer, to which 1 μg of IL-6 was added and incubated at 37° C. water bath for 24 h and 48 h, respectively. After that, the mixture was centrifuged, and 200 μL of a supernatant was taken, added with 50 μL of 5×protein loading buffer, denatured at 100° C. for 5 min and subjected to electrophoresis analysis.

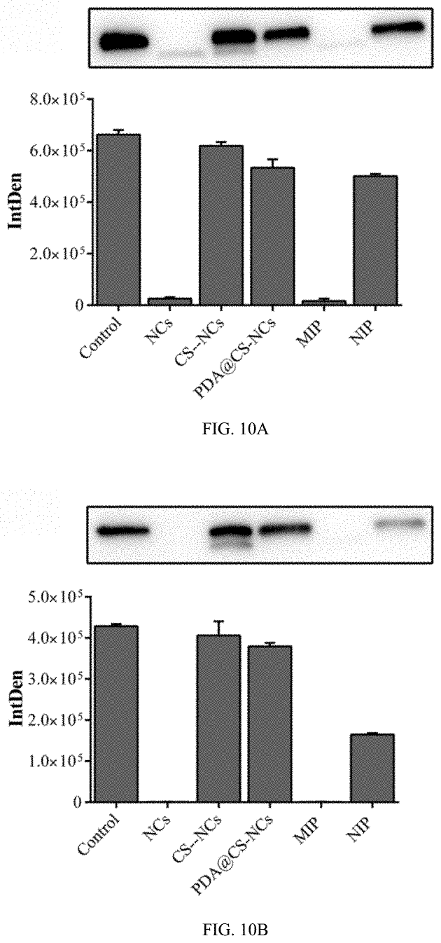

FIG. 10A showed electrophoretograms and gray values of the hydrolysis product of IL-6 after exposed to the five nanoparticles for 24 h. According to the electrophoresis results, compared to the Control group, most of the IL-6 in the NCs group and the MIP group was hydrolyzed, which was significantly different from the rest groups. According to the gray values, compared to the Control group, the gray value of the CS-NCs group was slightly decreased, suggesting that the chitosan had a masking effect on the NCs to prevent the IL-6 from being hydrolyzed. The gray value of the PDA@CS-NCs group was lower than that of the CS-NCs group, which was possibly because that some chitosan was removed during the elution to result in a partial exposure of the NCs. The gray value of the NIP group was similar to that of the PDA@CS-NCs group. Theoretically, the gray value of the PDA@CS-NCs group should be similar to the gray value of the CS-NCs group. Whereas, the gray value of the PDA@CS-NCs group was actually lower than the gray value of the CS-NCs group. It was probably because that due to a large specific surface area of the CS-NCs, the polydopamine film polymerized by dopamine was uneven or too thin, resulting in the exposure of the NCs.

FIG. 10B showed electrophoretograms and gray values of the hydrolysis product of IL-6 after exposed to the five nanoparticles for 48 h, and an overall trend thereof was consistent with that of FIG. 10A, but the electrophoretic band of the NIP group became shallower. In addition, the gray value of the NIP group here was half of the gray value of the PDA@CS-NCs group here, but both were significantly higher than the gray value of the MIP group here. In general, the hydrolysis of the IL-6 catalyzed by the MIP was due to a selective recognition by an imprinted site formed by the MIP The IL-6 entered the imprinted site to be exposed to the NCs, so as to be hydrolyzed into small fragments.

### Example 6 Cytotoxicity Assay by Methyl Thiazolyl Tetrazolium (MTT)

To a 96-well plate were inoculated 2×10⁴/mL RAW264.7 cells, 200 μL for per well and cultivated for 24 h. A supernatant was discarded after 24 h. Different concentrations of MIP solution dispersed in Dulbecco's Modified Eagle Medium (DMEM) were added into a MIP group. Different concentrations of NIP solution dispersed in DMEM were added into a NIP group. DEME was added into a Control group. After 24 h of cultivation, these groups were respectively added with 20 μL of 5 mg/mL MTT solution and then cultivated for 4 h. A supernatant was absorbed. These groups were respectively added with 180 μL of Dimethyl sulfoxide (DMSO) immediately to vibrate for 10 min. Then an absorbance of samples at 490 nm was measured by means of the microplate reader.

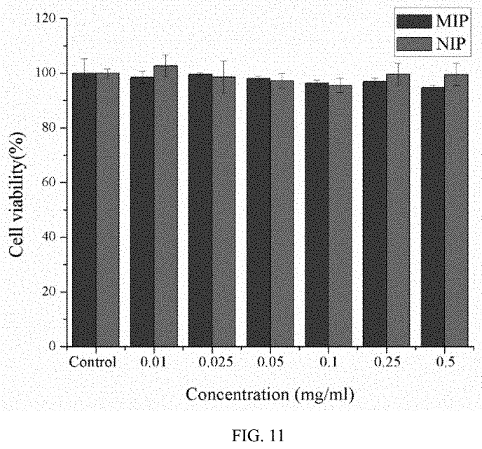

As shown in FIG. 11, the MIP/NIP was not toxic to the RAW264.7 cells.

### Example 7 Establishment of Lipopolysaccharide (LPS)-Induced Macrophage RAW264.7 Inflammation Model

To a 96-well plate were inoculated 2×10⁴/mL RAW264.7 cells, 200 μL for per well. A Control group and a model group (LPS group) were set up, 3 repeated wells for each group. Different concentrations of LPS in the LPS group included 100 ng/mL, 500 ng/mL, 1 μg/mL, 2 μg/mL and 5 μg/mL. The 96-well plate after inoculated was placed in an incubator and subjected to cell adhesion for 4 h. A supernatant was discarded. 200 μL of blank medium were added into the Control group. 200 μL of different concentrations of LPS were added into the LPS group to cultivate for 24 h. After the cultivation, the supernatant was taken into an inactivated EP tube to centrifuged (1000 rpm, 4° C. for 5 min). A supernatant was taken to test a NO level and an IL-6 level by strictly following the operating steps of NO assay kit and IL6 ELISA KIT.

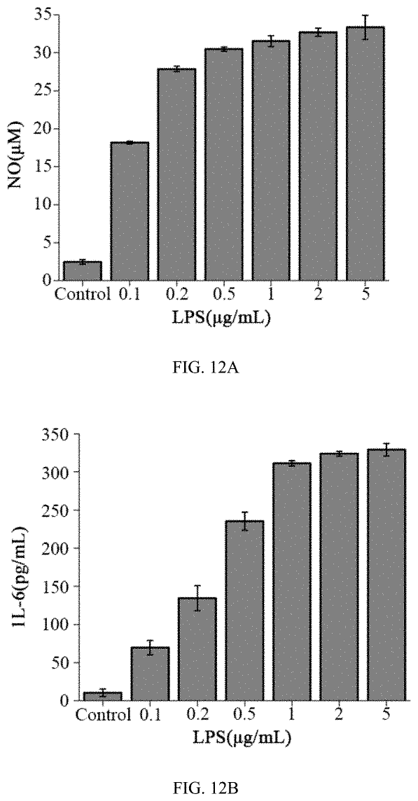

As shown in FIG. 12A, after 24 h treatment by LPS, the NO level in the macrophage was significantly increased, and with an increased LPS concentration came an increased NO level. Nevertheless, when the LPS concentration reached to 1 g/mL, the NO level was not increased, so was those higher concentration LPS groups. As shown in FIG. 12B, after 24 h treatment by LPS, the IL-6 level in the macrophage was increased, and with an increased LPS concentration came an increased IL-6 level. Nevertheless, when the LPS concentration reached to 1 μg/mL, the IL-6 level was not increased. Accordingly, 1 μg/mL of LPS was suitable to treat the RAW264 0.7 cells for a macrophage inflammation model.

### Example 8 Hydrolysis of IL-6 by MIP Co-Cultured with Cells

To a 96-well plate were inoculated 3.5×10⁵/mL RAW264.7 cells, 200 μL for per well, followed by cell adhesion for 4 h. A supernatant was discarded. The mixture was added with 200 μL of 1 μg/mL LPS to incubate for 24 h. A Control group, a model group (LPS group), a MIP group and a NIP group were set up, 3 repeated wells for each group. A concentration of MIP in the MIP group and a concentration of NIP in the NIP group were 0.1 mg/ml, 0.05 mg/ml, 0.025 mg/ml and 0.01 mg/ml. The Control group and the LPS group were respectively added with 50 μL of DEME. 50 μL of MIP solution dispersed a corresponding concentration of MIP in DEME were added into the MIP group. 50 μL of NIP solution dispersed a corresponding concentration of NIP in DEME were added into the NIP group. And these groups were cultured for 2 h, 4 h, 10 h and 24 h. A cell supernatant was collected and centrifuged (1000 rpm, 5 min) and a supernatant was taken.

The IL-6 level in the supernatant was detected by strictly following the operating steps of the IL-6 ELISA KIT.

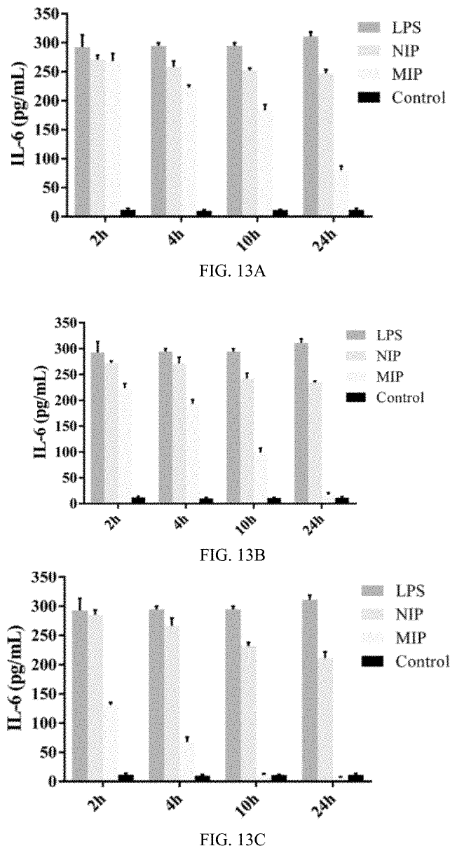

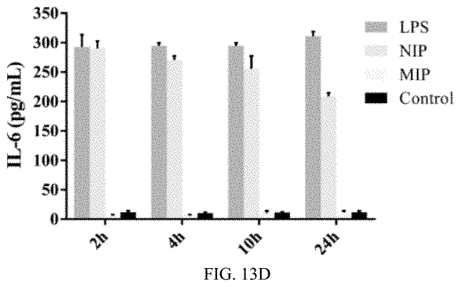

As shown in FIGS. 13A-13D, when the concentration of MIP was 0.01 mg/ml, the MIP-catalyzed hydrolysis effect of IL-6 was related to time, and with a longer reaction time came a better effect. The IL-6 was hydrolyzed 70% after 24 h. With a higher concentration of MIP came a better hydrolysis efficiency. When the concentration of MIP was 0.01 mg/ml, the IL-6 was hydrolyzed completely after 2 h. A small amount of IL-6 was hydrolyzed by the NIP. A higher concentration of NIP, the more IL-6 was hydrolyzed. Whereas, there was a significant difference between the NIP and MIP. The MIP was significantly hydrolyzed the IL-6, suggesting that in a cellular level, the MIP had the effect of catalyzing the hydrolysis of the inflammatory cytokine IL-6 secreted in the inflammatory response.

### Example 9 In-Vivo Catalyzed Hydrolysis

70 C57BL/6 male mice, aged 7-8-weeks and weighing 20-25 g, were purchased from Zhuhai Biotest Biotechnology Co. and adopted at SPF class animal room, Laboratory Animal Center, School of Traditional Chinese Medicine, Guangzhou University of Chinese Medicine, a license number is SYXK( [symbol] )201900202.

6 7-8-week-old male Balb/nude mice, weighing 20-25 g were purchased from Zhuhai Biotest Biotechnology Co. and adopted at SPF class animal room, Laboratory Animal Center, School of Traditional Chinese Medicine, Guangzhou University of Chinese Medicine, a license number is SYXK( [symbol] )201900202.

### 1. Evaluation of Immunogenicity of MIP

A Control group and a MIP group were set up, each for 10 C57BL/6 mice. The Control group was subjected to tail vein injection with 200 μL of normal saline. The MIP group was subjected to tail vein injection with 0.05 g/g MIP (MIP was dispersed in normal saline) according to weight of mice. 24 h after the injection, the heart, liver, spleen, lungs and kidneys of mice were taken by dissection after orbital blood sampling, followed by fixation with 4% paraformaldehyde.

(1) Blood Routine Examination

Heparin sodium anticoagulated whole blood was fully analyzed by Automatic Veterinary Hematology Analyzer of Mindray.

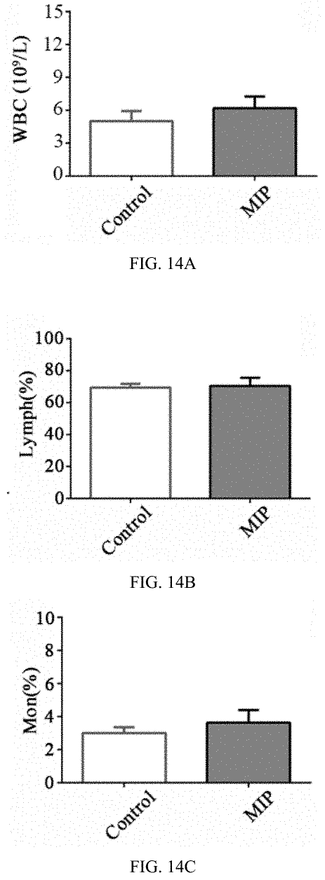

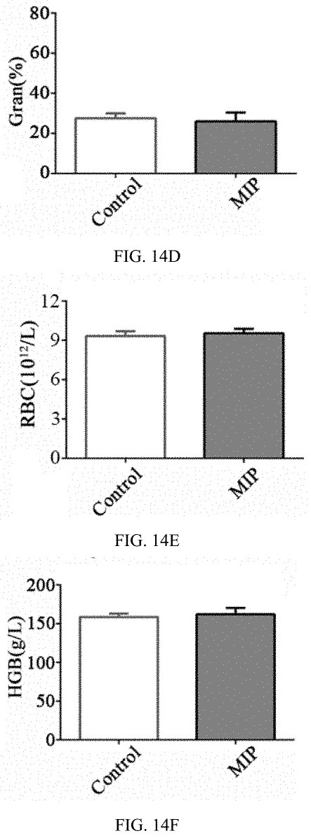

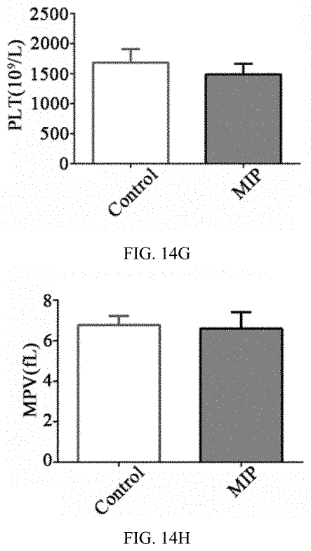

Results were shown in FIGS. 14A-14H. Hematological parameters were white blood cell count (WBC), a percentage of lymphocytes (Lymph %), a percentage of monocytes (Mon %), a percentage of neutrophilic granulocyte (Gran %), red blood cell count (RBC), hemoglobin (HGB), platelet count (PLT) and mean platelet volume (MPV). There was no significant difference in all hematological parameters in the MIP group compared to the Control group (P>0.05), suggesting that the MIP had no immunogenicity to mice.

(2) Test of Serum Biochemistry and Inflammatory Cytokine

A blood specimen was placed at room temperature for 2 h and then centrifuged at 4° C. for 30 min at 3000 rpm/min. A supernatant serum was dividedly packed and then stored at −80° C. The serum was taken out to test biochemical parameters according to the kit instructions. The biochemical parameters were alanine aminotransferase (ALT), aspartate aminotransferase (AST), blood urea nitrogen (BUN), creatinine (CREA) and urea (UA). The serum should be centrifuged again after thawed to test the serum biochemistry.

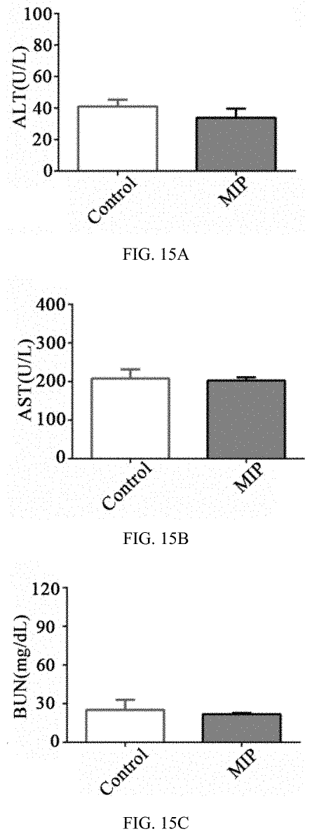

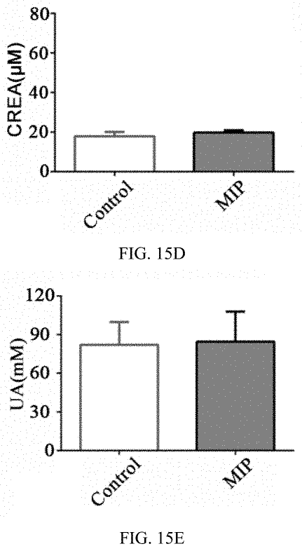

Results were shown in FIGS. 15A-15E. Liver function parameters were ALT and AST; renal function parameters were BUN, CREA and UA. There was no significant difference in the liver and renal function parameters in the MIP group compared to the Control group (P>0.05), suggesting that the MIP had no hepatic or renal impairment effect on mice.

The serum stored at −80° C. was taken out to test the IL-6, IL-8 and TNF-α according to the kit instructions.

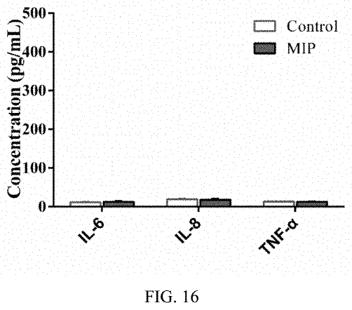

Results were shown in FIG. 16. Compared to the Control group, the inflammatory cytokine level of the MIP group was not increased and consistent with the Control group (P>0.05), suggesting that the MIP was not toxic and did not activate relevant immune cells to secrete relevant inflammatory cytokines.

### 2. Administration

(1) Preparation of Models

60 adult male C57BL/6 mice were divided into a Control group, a LPS group (model group), a DXM group (an administration dose was 0.1 mg per mouse), a HNE group (an administration dose was 5 μg/per mouse), a low-dose MIP group (L-MIP group, an administration dose was 0.05 g/g) and a high-dose MIP group (H-MIP group, an administration dose was 0.2 g/g).

The Control group was intraperitoneally injected with an equal volume of 0.9% normal saline.

The LPS group was intraperitoneally injected with 20 mg/kg LPS solution.

With respect to the DXM group, one shot of dexamethasone sodium phosphate injection (5 mg/mL, specification of 2 mL, by intramuscular injection) was diluted to a concentration of 1 mg/mL with filtered and sterilized 5% glucose solution. 20 mg/kg LPS solution was intraperitoneally injected. 1 h later, the dexamethasone sodium phosphate injection was intramuscularly injected at a dose of 0.1 mL/20 g per mouse.

The L-MIP group was intraperitoneally injected with 20 mg/kg LPS solution. 1 h later, 0.05 g/g MIP were injected by tail vein injection.

The H-MIP group was intraperitoneally injected with 20 mg/kg LPS solution. 1 h later, 0.2 g/g MIP were injected by tail vein injection.

The HNE group was intraperitoneally injected with 20 mg/kg LPS solution. 1 h later, HNE solution were injected by tail vein injection with 5 μg per mouse.

For the six groups, 24 h after the injection, the heart, liver, spleen, lungs and kidneys of mice were taken by dissection after orbital blood sampling

The blood routine examination and test of serum biochemistry and inflammatory cytokine were performed as the foregoing.

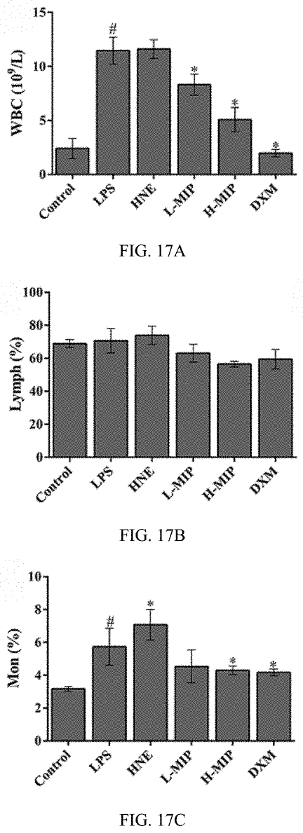

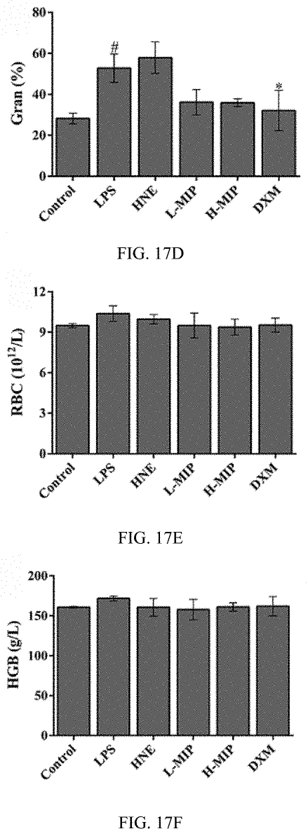

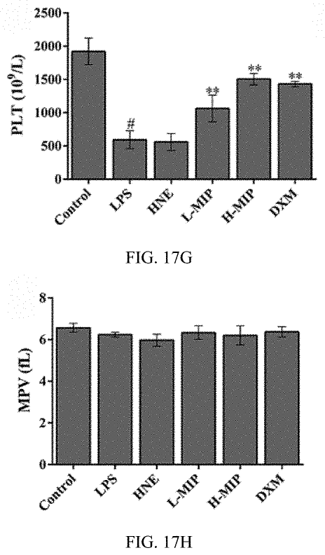

Results of the blood routine examination were shown in FIGS. 17A-17H. Hematological parameters included WBC, Lymph %, Mon %, Gran %, RBC, HGB, PLT and MVP. 24 h after modeled, a WBC, Mon % and Gran % in inflammatory cells of the LPS group were significantly upregulated compared to that of the Control group (P<0.05), but a Lymph % had no significant change (P>0.05). Erythrocyte parameters RBC and HGB of the LPS group had no significant change (P>0.05). A PLT of the LPS group was significantly changed (P<0.05) and a MPV of the LPS group had no significant change (P>0.05) compared to those of the Control group, suggesting that the high-dose LPS induced a systemic acute inflammatory response in mice, the number of inflammatory cells was dramatically increased and no significant effect was occurred on the erythrocyte parameters. Upon successfully modeled the LPS group, free enzymes were administrated to the HNE group. A Mon % of the HNE group was significantly changed (P<0.05) while other parameters were not (P>0.05) compared to those of the LPS group, suggesting that instead of suppressing or reducing, the HNE further promoted the systemic inflammatory response. After 24 h of intravenous administration of MIP, a WBC in inflammatory cells of the L-MIP group was significantly reduced compared to that of the LPS group (P<0.05). A Lymph %, Mon % and Gran % of the L-MIP group had no significant change compared to those of the LPS group (P>0.05). A PLT of the L-MIP group was significantly increased (P<0.01) while other parameters were not (P>0.05) compared to those of the LPS group. After 24 h of intravenous administration of MIP, compared to those of the LPS group, a WBC and Mon % in inflammatory cells of the H-MIP group were significantly changed (P>0.05) and the PLT was significantly changed (P<0.01), while other parameters were not (P>0.05). After 24 h of administration, the MIP brought a decline in the number and proportion of inflammatory cells, suggesting that inflammatory symptoms were reduced, which was consistent with an observation that the MIP group was in a better physiological state than the LPS group 24 h after drug administration. After 24 h of drug administration, the mice in the LPS group were shivered and had a lusterless coat color, secretions around eyes, poor spirit, poor mobility and cold body. The mice in the L-MIP group also had the above-mentioned symptoms, but with a slightly shiny coat and improved mobility. Differently, the mice in the H-MIP group had good spirit, active behavior, shiny coat and no secretion around the eyes. The results suggested that as the administration level of MIP increased, the treating effect on inflammatory symptoms in mice was enhanced, and the H-MIP group showed a better effect in reducing the inflammatory response. A dexamethasone sodium phosphate injection was used as a positive drug, which was intramuscularly injected. The DXM showed a great and wide-range anti-inflammatory effect, and therefore was usually chosen as an anti-inflammatory drug in the acute phase. After 24 h of DXM administration, compared to those of the LPS group, a WBC, Mon % and Gran % in inflammatory cells of the DXM group were significantly downregulated (P<0.05), a PLT of the DXM group was significantly downregulated (P<0.01) while other parameters were not changed (P>0.05), suggesting that the DXM showed a great anti-inflammatory effect and an efficacy thereof was better than that of the L-MIP group and the H-MIP group.

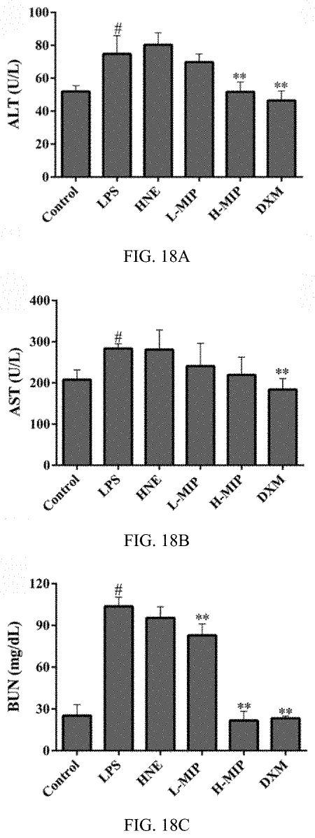

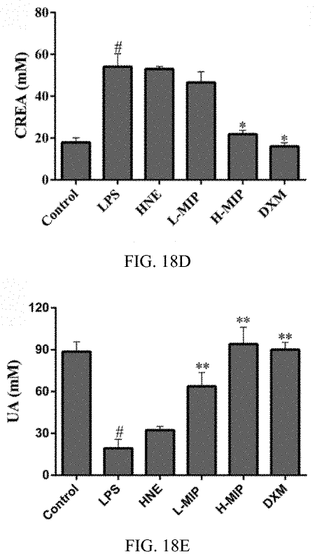

Results of serum biochemistry were shown in FIGS. 18A-18E. Liver function parameters were ALT and AST. Renal function parameters were BUN, CREA and UA. 24 h after modeled, an ALT, AST, BUN and CERA of the LPS group were significantly increased (P<0.05) and an UA of the LPS group was significantly decreased (P<0.05), suggesting that hepatic and renal impairments were occurred when the mice were stimulated by the LPS. Compared to those of the LPS group, the parameters of the HNE group showed no significant difference, suggesting that sever hepatic and renal impairments were occurred. Compared to those of the LPS group, an ALT and AST in the liver function parameters of the L-MIP group showed no difference (P>0.05), a BUN and UA in the renal function parameters showed a significant difference (P<0.01) and a CREA in the renal function parameters showed no significant difference (P>0.05). Accordingly, after 24 h of drug administration, the hepatic impairment of the L-MIP group was significantly diminished, but the renal function was still severely impaired. Compared to those of the LPS group, an ALT in the liver function parameters of the H-MIP group was significantly upregulated (P<0.01) while an AST of the H-MIP group showed no significant change. A CREA (P<0.01) and UA (P<0.05) in the renal function parameters of the H-MIP group showed significant difference, suggesting that high-dose MIP could improve the hepatic and renal impairments. Compared to those of the LPS group, an ALT, AST, BUN, UA and CERA of the DXM group showed significantly difference, suggesting that DXM had a good anti-inflammatory effect and there was no significant hepatic or renal impairments after DXM administration.

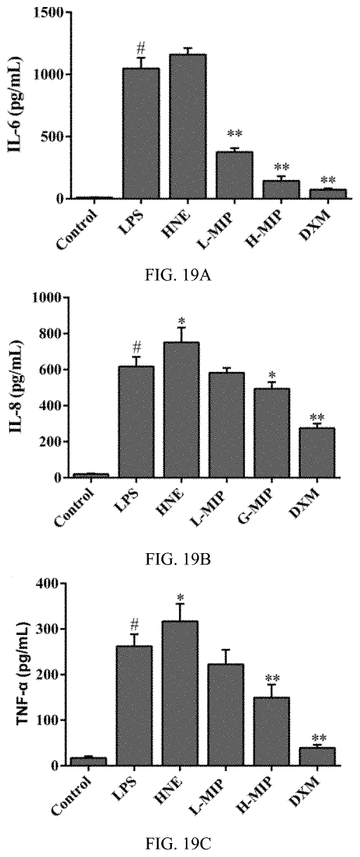

Results of inflammatory cytokine were shown in FIGS. 19A-19C. Compared to those of the Control group, a large number of cytokines were secreted in the LPS group, and the IL-6 level, IL-8 level and TNF-α level were significantly upregulated. After 24 h of administration, the IL-8 level and TNF-α level of the HNE group were upregulated (P<0.05). Compared to those of the LPS group, the IL-6 level of the L-MIP group was significantly downregulated (P<0.01), the TNF-α level of the L-MIP group was slightly decreased, but the IL-8 level and TNF-α level of the L-MIP group had no significant change (P>0.05). After administration, the IL-6 level of the H-MIP group was significantly downregulated (P<0.01) to lower than the IL-6 level of the L-MIP group, and the IL-8 level (P<0.05) and TNF-α level (P<0.01) of the H-MIP group were significantly decreased with a smaller amount. The MIP was capable of selectively catalyzing the hydrolysis of IL-6. The L-MIP and the H-MIP catalyzed the hydrolysis of LPS to induce a production of a large amounts of IL-6. Since the MIP was capable of catalyzing the hydrolysis, after 24 h of MIP administration the IL-6 level was significantly decreased, and with a higher concentration of MIP came a better hydrolysis effect. With a low-dose MIP, the IL-8 level and TNF-α level showed no significant change, because the low-dose MIP could merely catalyze the hydrolysis of IL6. With a high-dose MIP, in addition to the IL-6 level, the IL-8 level and TNF-α level were also downregulated. This might because the high-dose MIP could rapidly catalyze the hydrolysis of the IL-6. Due to an inter-cytokine regulation, through an inter-cytokine signaling, it was the significant downregulation of IL-6 level which contributed to the downregulation of the IL-8 and TNF-α, rather than the hydrolysis of IL-8 and TNF-α. After the DXM administration, compared to those of the LPS group, the IL-6, IL-8 and TNF-α of the DXM group were significantly downregulated (P<0.01) and a downregulation effect of the DXM group was better than a downregulation effect of the H-MIP group, suggesting that the DXM was a good anti-inflammatory drug which regulated the inflammatory response and normalized the cytokine level.

### 3. In-Vivo Imaging

20 mg of DIR powder was dissolved in ethanol absolute to obtain a 5 mM DIR concentrated solution. 10 μL of 5 mM DIR concentrated solution were added to 2 mL of PBS to obtain a free DIR solution.

6 mL of PBS containing 0.5 mg/mL BSA were added to 30 μL of 5 mM DIR concentrated solution and vibrated 30 min at room temperature away from light. The solution was added with 40 μL of 120 mM aqueous CuSO₄ solution and stirred for 2 h at 37° C. away from light followed by centrifugation (10000 rpm, 5 min) and washing. A supernatant was discarded to obtain NCs. The NCs were dispersed into 1.15 mg/mL CS solution (pH 6.2) and stirred for 30 min away from light followed by centrifugation (10000 rpm, 5 min) and washing. A supernatant was discarded to obtain CS-NCs. The CS-NCs was dispersed into 20 mM Tris-HCl (pH 8.0) and added with 0.1 mg/mL dopamine solution to polymerize for 3 h at 37° C. away from light. Then the mixture was subjected to centrifugal washing twice with deionized water (10000 rpm, 5 min) and centrifugal washing 3 times with ethanol absolute. A precipitate was dispersed into 2 mL of PBS to obtain a DIR-carried PDA@CS-NCs.

6 7 to 8-week-old male Balb/nude mice were divided into a free DIR group and a DIR-carried PDA@CS-NCs group. The free DIR group was subjected to tail vein injection with 200 μL of free DIR solution. The DIR-carried PDA@CS-NCs group was subjected to tail vein injection with 200 μL of DIR-carried PDA@CS-NCs solution. An in-vivo imaging was performed in the two groups after 1 h, 10 h, 24 h and 48 h of drug injection. 48 h later, these nude mice were executed and dissected to obtain tissues. The tissues were imaged by Berthold IndiGo Ver.A01.19.01 with excitation/emission of 630 nm/680 nm, to study a distribution of the free DIR and a distribution of the DIR-carried PDA@CSNCs in mice and an accumulation and retention of the free DIR and the DIR-carried PDA@CSNCs in organs.

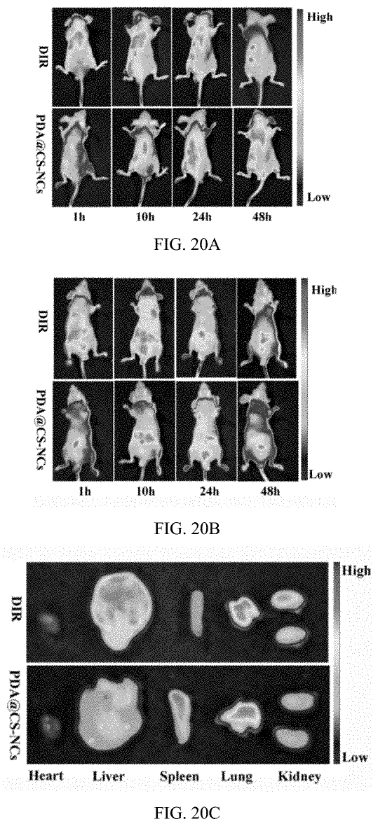

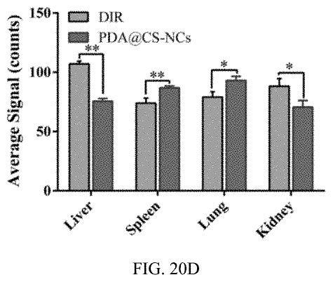

FIG. 20A showed a fluorescence imaging of a back of nude mice for monitoring the distribution of the free DIR in nude mice after intravenous injection of the free DIR solution and the DIR-carried PDA@CS-NCs solution with small animal in-vivo imager. FIG. 20B showed a fluorescence imaging of an abdomen of nude mice for monitoring the distribution of the free DIR in nude mice after intravenous injection of the free DIR solution and the DIR-carried PDA@CS-NCs solution with small animal in-vivo imager. FIGS. 20C-D showed the level of the free DTR and the DTR-carried PDA@CSNCs in different organs. The free DIR group was taken as a control group. Fluorescent images at different time intervals were acquired with the in-vivo imaging system. As shown in FIGS. 20A and 20B, the free DIR was distributed faster in vivo and was distributed to the whole body by 1 h of intravenous injection. A less distribution of the free DIR in head and neck at 48 h indicated that the free DIR was distributed and metabolized faster. The DIR-carried PDA@CSNCs was distributed slow and uneven in vivo by 1 h of intravenous injection. The DIR-carried PDA@CSNCs was distributed to the whole body by 10 h and remained distributed to the whole body by 48 h, suggesting that the DTR-carried PDA@CSNCs were distributed slower compared to the free DIR, but could remain for a longer time. Therefore, the DIR-carried PDA@CSNCs had a longer retention time in vivo and was not rapidly metabolized. Mice were dissected 48 h after injection and subjected to fluorescence imaging of isolated organs (heart, liver, spleen, lung and kidney) by IndiGo. As shown in FIGS. 20C and 20D, a fluorescence intensity of the free DIR group was significantly stronger than that of the DIR-carried PDA@CSNCs group in the liver and kidney, whereas a fluorescence intensity of the free DIR group was significantly stronger than that of the free DTR group in the spleen and lung, namely, the free DTR group had a faster metabolism.

In summary, the nanoparticle provided herein has a core (nanozyme)-shell (template protein-imprinted polymer) structure. The nanoparticle has excellent applicability, and can regulate the excessively-secreted cytokines or proteins to enable a better therapeutic effect and relieve the side effect of drugs. Moreover, the nanoparticle has simple preparation. The nanoparticle can specifically identify and bind cytokines, and further catalyze the hydrolysis of the cytokines. The nanoparticle provided herein can be employed in the preparation of drugs for the treatment of CRS, and thus has broad application prospects.

Described above are merely preferred embodiments of the disclosure, which are illustrative and are not intended to limit the disclosure. It should be understood that any variations, modifications and replacements made by those skilled in the art without departing from the spirit and scope of the disclosure should fall within the scope of the disclosure defined by the appended claims.

What is claimed is:

1. A nanoparticle for specifically hydrolyzing a template protein, wherein the nanoparticle comprises a nanozyme as a core and a template protein-molecularly imprinted polymer as a shell; and
a particle size of the nanoparticle is 1 nm-50 μm.

2. The nanoparticle of claim 1, wherein the particle size of the nanoparticle is 100 nm-5 μm.

3. The nanoparticle of claim 1, wherein an enzyme of the nanozyme is serine proteinase;
the serine proteinase is human neutrophil elastase, cathepsin G, protease 3 or a combination thereof, and
the nanozyme further comprises a water-insoluble carrier.

4. The nanoparticle of claim 3, wherein the water-insoluble carrier is an inorganic salt crystal;
the inorganic salt crystal is a copper phosphate crystal, a calcium hydrogen phosphate crystal or a combination thereof; and
the nanozyme has an inorganic hybrid nanoflower structure formed by hybridization of the enzyme with the inorganic salt crystal.

5. The nanoparticle of claim 3, wherein a raw material for preparing the template protein-molecularly imprinted polymer comprises an organic polymer material and the template protein;
the organic polymer material is a positively-charged amino-rich material; and
the template protein is a cytokine, a coagulation factor, an immunoglobulin, a complement or a protein from an extracellular matrix.

6. The nanoparticle of claim 5, wherein the organic polymer material is a water-soluble and positively-charged amino-rich polysaccharide;
the protein from the extracellular matrix is collage, elastin, fibrin, fibronectin or a combination thereof;
the cytokine is interleukin, interferon, tumor necrosis factor superfamily, colony-stimulating factor, chemokine, growth factor or a combination thereof; and
the interleukin is interleukin-6 (IL-6), IL-2, IL-8 or a combination thereof.

7. The nanoparticle of claim 5, wherein the raw material for preparing the template protein-molecularly imprinted polymer further comprises dopamine.

8. The nanoparticle of claim 4, wherein the shell of the nanoparticle is a polydopamine layer wrapped on a surface of the nanozyme; and there is a cavity between the nanozyme and the polydopamine layer.

9. The nanoparticle of claim 8, wherein the nanozyme has a nanoflower structure formed by hybridization of the human neutrophil elastase with the copper phosphate crystal.

10. The nanoparticle of claim 1, wherein a surface of the nanoparticle is provided with a targeted modification material; and
the targeted modification material is polyethylene glycol.

11. A method for preparing the nanoparticle of claim 1, comprising:
coating the template protein-molecularly imprinted polymer on a surface of the nanozyme to form the nanoparticle with a core-shell structure.

12. The method of claim 11, wherein the step of “coating the template protein-molecularly imprinted polymer on a surface of the nanozyme” comprises:
coating an organic polymer material layer on the surface of the nanozyme; and
allowing the template protein to be adsorbed to a surface of the organic polymer material layer; and preparing a polydopamine layer on the organic polymer material layer by polymerization.

13. The method of claim 12, further comprising:
after the polydopamine layer is prepared, removing the template protein and the organic polymer material layer to form a cavity between the nanozyme and the polydopamine layer.

14. The method of claim 11, wherein a preparation of the nanozyme comprises:
subjecting a human neutrophil elastase (HNE) and a copper phosphate crystal to hybridization to form the nanozyme with an inorganic hybrid nanoflower structure.

15. The method of claim 14, wherein the step of “subjecting a human neutrophil elastase (HNE) and a copper phosphate crystal to hybridization” is performed through steps of:
reacting the HNE with an aqueous copper sulphate solution in a phosphate buffered saline (PBS) containing bovine serum albumin (BSA) followed by solid-liquid separation to collect the nanozyme.

16. The method of claim 11, wherein the step of “coating the template protein-molecularly imprinted polymer on a surface of the nanozyme” is performed through steps of:
dispersing the nanozyme in a chitosan solution to obtain a chitosan-coated nanozyme;

17. The method of claim 11, further comprising:
subjecting a surface of the nanoparticle to targeted modification.

18. A method for treating cytokine release syndrome in a subject in need thereof, comprising:
administering to the subject a therapeutically effective amount of the nanoparticle of claim 1.
